(19) United States
(12) Patent Application Publication
(10) Pub. No.: US 2016/0095581 A1
(43) Pub. Date: Apr. 7, 2016

(54) ULTRASONIC DIAGNOSIS APPARATUS

(71) Applicants: Kabushiki Kaisha Toshiba, Minato-ku (JP); Toshiba Medical Systems Corporation, Otawara-shi (JP)

(72) Inventors: Naoki YONEYAMA, Otawara (JP); Kouji Ando, Otawara (JP); Minori Izumi, Shioya (JP); Nobuhide Oi, Nasushiobara (JP); Taku Muramatsu, Nasushiobara (JP)

(73) Assignees: Kabushiki Kaisha Toshiba, Minato-ku (JP); Toshiba Medical Systems Corporation, Otawara-shi (JP)

(21) Appl. No.: 14/963,793

(22) Filed: Dec. 9, 2015

Related U.S. Application Data
(63) Continuation of application No. PCT/JP2014/003100, filed on Jun. 10, 2014.

(30) Foreign Application Priority Data
Jun. 11, 2013 (JP) 2013-122693

(51) Int. Cl. A61B 8/08; A61B 8/00

(52) U.S. Cl. CPC A61B 8/5215; A61B 8/466; A61B 8/0883; A61B 8/463; A61B 8/4444; A61B 8/4254

(57) ABSTRACT

An ultrasonic diagnosis apparatus of an embodiment includes: an ultrasonic image generation section that generates an ultrasonic image based on a reception signal from an ultrasonic probe; a position information acquisition section that acquires position information on a three-dimensional space of the ultrasonic probe; an image acquisition section that obtains image data and acquires a reference image corresponding to the ultrasonic image based on the image data; a reference image forming section that identifies a to-be-displayed cross section orientation of the acquired reference image and forms a reference image which a cross section orientation is identified; and a display section that displays a formed reference image by the reference image forming section and ultrasonic image formed by the ultrasonic image generation section.

### CROSS-REFERENCE TO RELATED APPLICATION

This application is a continuation of International Application No. PCT/JP2014/003100, filed on Jun. 10, 2014, which is based upon and claims the benefit of priority from the prior Japanese Patent Application No. 2013-122693, filed on Jun. 11, 2013, the entire contents of which are incorporated herein by reference.

### FIELD

An embodiment described below relates to an ultrasonic diagnosis apparatus and, more particularly, to an ultrasonic diagnosis apparatus that displays, as a reference image, an image that a medical image processing apparatus acquires together with an ultrasonic image.

### BACKGROUND

Conventionally, an ultrasonic diagnosis apparatus has been used as a medical apparatus. The ultrasonic diagnosis apparatus can be connected to various modalities such as an X-ray CT apparatus (X-ray computed tomography apparatus) and an MRI apparatus (magnetic resonance imaging apparatus) over an in-hospital network and supports diagnosis and treatment of disease by utilizing an ultrasonic image acquired thereby and an image acquired from another medical image diagnosis apparatus.

For example, there is known an ultrasonic diagnosis apparatus that aligns a cross section to be scanned by an ultrasonic probe and a CT image or an MRI image in which a lesion is detected by using a magnetic position sensor and displays a CT or MRI image of the same cross-section as that of an ultrasonic image (echo image) as a reference image, so as to navigate the ultrasonic probe to a position corresponding to the lesion.

The function of thus displaying the aligned and combined ultrasonic image (echo image) and reference images (hereinafter, referred to as “fusion” function) is now essential in diagnosis of early cancer. Note that the magnetic position sensor is provided in a magnetic field formed by, e.g., a transmitter and is mounted to the ultrasonic probe.

Conventionally, in the alignment between the echo image and reference image, images in reference cross-section orientations such as an “axial” image, a “sagittal” image, and a “coronal” image are displayed as reference images, and the ultrasonic image is aligned with these reference images. However, an optimum cross section differs depending on a part to be diagnosed, so that it inconveniently takes a lot of effort to adjust the reference image. Further, the optimum cross section differs also depending on a type of the probe, thus requiring a lot of effort to adjust the reference image.

### BRIEF DESCRIPTION OF THE DRAWINGS

FIG. 1 is a block diagram illustrating a schematic configuration of an ultrasonic diagnosis apparatus according to an embodiment;

FIG. 2 is an explanatory view illustrating an arrangement of a position sensor of a position information acquisition section in the embodiment;

FIGS. 3A and 3B are explanatory views illustrating, respectively, display examples of a reference and ultrasonic images in the embodiment;

FIG. 4 is a block diagram illustrating a configuration of a CPU and components around the CPU in the embodiment;

FIG. 5 is an explanatory view schematically illustrating cross section orientations in the embodiment;

FIG. 6 is an explanatory view illustrating an examination of a prostate gland in the embodiment;

FIGS. 7A to 7C are explanatory views illustrating general rotating processing of the reference image;

FIGS. 8A and 8B are explanatory views illustrating the rotation processing of the reference image in the embodiment;

FIG. 9 is an explanatory view illustrating an example of a scanning operation performed for an abdominal area and a heart by the probe in the embodiment;

FIG. 10 is an explanatory view schematically illustrating an apical four-chamber cross section in the embodiment; and

FIG. 11 is a flowchart explaining operation of a CPU in the embodiment.

### DETAILED DESCRIPTION

An ultrasonic diagnosis apparatus according to an embodiment includes: an ultrasonic image generation section that generates an ultrasonic image based on a reception signal from an ultrasonic probe that transmits an ultrasonic wave to a subject and receives the ultrasonic wave from the subject; a position information acquisition section that includes a position sensor mounted to the ultrasonic probe and acquires position information on a three-dimensional space of the ultrasonic probe; an image acquisition section that obtains image data and acquires a reference image corresponding to the ultrasonic image based on the image data; a reference image forming section that identifies a to-be-displayed cross section orientation of acquired the reference image according to at least one of information related to a examination purpose for the subject and information related to a type of the ultrasonic probe, and forms a reference image which a cross section orientation is identified; and a display section that displays a formed reference image by the reference image forming section and ultrasonic image formed by the ultrasonic image generation section.

### First Embodiment

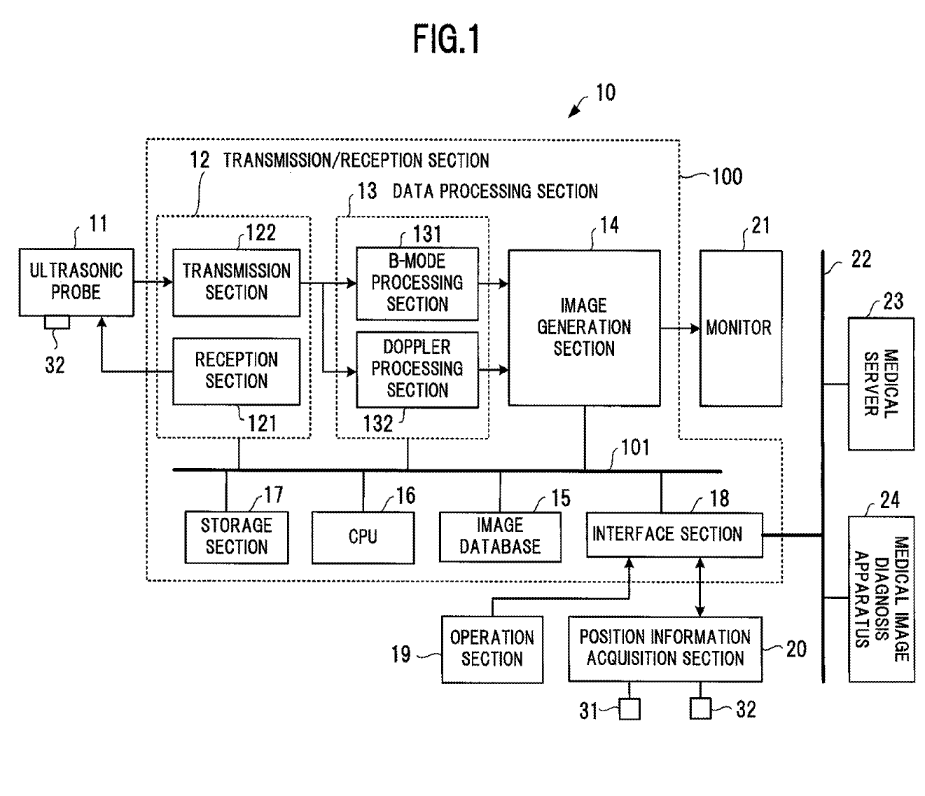

FIG. 1 is a block diagram illustrating a schematic configuration of an ultrasonic diagnosis apparatus 10 according to an embodiment. As illustrated in FIG. 1, a main body 100 of the ultrasonic diagnosis apparatus 10 includes an ultrasonic probe 11 that transmits an ultrasonic wave to a subject (not illustrated) and receives the ultrasonic wave from the subject, a transmission/reception section 12 that drives the ultrasonic probe 11 to perform ultrasonic scanning for the subject, and a data processing section 13 that processes a reception signal acquired by the transmission/reception section 12 to generate image data such as B-mode image data and Doppler image data.

The main body 100 further includes an image generation section 14 that generates two-dimensional image data based on the image data output from the data processing section 13 and an image database 15 that collects and stores the image data generated by the image generation section 14. The main body 100 further includes a central processing unit (CPU) 16 that controls the entire apparatus, a storage section 17, and an interface section 18 that connects the main body 100 to a network 22. The interface section 18 is connected with an operation section 19 through which various command signals and the like are input and a position information acquisition section 20. The main body 100 is connected with a monitor (display section) 21 that displays the image and the like generated by the image generation section 14. The CPU 16 and the above circuit sections are connected via a bus line 101.

The interface section 18 can be connected to the network 22, allowing the image data obtained by the ultrasonic diagnosis apparatus 10 to be stored in an external medical server 23 over the network 22. The network 22 is connected with a medical image diagnosis apparatus 24 such as an MRI apparatus, an X-ray CT apparatus, or a nuclear medical diagnosis apparatus, allowing medical image data obtained by the medical image diagnosis apparatus 24 to be stored in the medical server 23.

The ultrasonic probe 11 transmits/receives an ultrasonic wave while bringing a leading end face thereof into contact with a body surface of the subject and has a plurality of piezoelectric vibrators arranged in one dimension. The piezoelectric vibrator is an electro-acoustic conversion element, which converts an ultrasonic driving signal into a transmitting ultrasonic wave at transmission and converts a receiving ultrasonic wave from the subject into an ultrasonic receiving signal at reception. The ultrasonic probe 11 is, e.g., an ultrasonic probe of a sector type, of a linear type, or of a convex type. Hereinafter, the ultrasonic probe 11 is sometimes referred to merely as “probe”.

The transmission/reception section 12 includes a transmission section 121 that generates the ultrasonic driving signal and a reception section 122 that processes the ultrasonic receiving signal acquired from the ultrasonic probe 11. The transmission section 121 generates the ultrasonic driving signal and outputs it to the probe 11. The reception section 122 outputs the ultrasonic receiving signal (echo signal) acquired from the piezoelectric vibrators to the data processing section 13.

The data processing section 13 includes a B-mode processing section 131 that generates B-mode image data from the signal output from the transmission/reception section 12 and a Doppler processing section 132 that generates Doppler image data. The B-mode processing section 131 performs envelope detection for the signal from the transmission/reception section 12 and then performs logarithmic conversion for the signal that has been subjected to the envelop detection. Then, the B-mode processing section 131 converts the logarithmically converted signal into a digital signal to generate B-mode image data and outputs it to the image generation section 14.

The Doppler processing section 132 detects a Doppler shift frequency of the signal from the transmission/reception section 12 and then converts the signal into a digital signal. After that, the Doppler processing section 132 extracts a blood flow or tissue based on Doppler effect, generates Doppler data and outputs the generated data to the image generation section 14.

The image generation section 14 generates an ultrasonic image using the B-mode image data, Doppler image data, and the like output from the data processing section 13. Further, the image generation section 14 includes a DSC (Digital Scan Converter) and performs scanning and conversion of the generated image data to generate an ultrasonic image (B-mode image or Doppler image) that can be displayed on the monitor 21. Thus, the ultrasonic probe 11, transmission/reception section 12, data processing section 13, and image generation section 14 constitute an ultrasonic image generation section that generates the ultrasonic image.

The image database 15 stores the image data generated by the image generation section 14. Further, the image database 15 obtains, via the interface section 18, three-dimensional image data, e.g., an MPR image (multiple slices image), photographed by the medical image diagnosis apparatus 24 (MRI apparatus or X-ray CT apparatus) and stores the acquired three-dimensional image data. The acquired three-dimensional image data can be used for acquisition of a reference image (to be described later) corresponding to the ultrasonic image. Thus, the image database 15 and interface section 18 constitute an image acquisition section that acquires the three-dimensional image data.

The CPU 16 executes various processing while controlling the entire ultrasonic diagnosis apparatus 10. For example, the CPU 16 controls the transmission/reception section 12, the data processing section 13, and the image generation section 14 based on, e.g., various setting requests input through the operation section 19 or various control programs and various setting information read from the storage section 17. Further, the CPU 16 performs control so as to display the ultrasonic image stored in the image database 15 on the monitor 21.

The storage section 17 stores various data such as a control program for performing ultrasonic wave transmission/reception, image processing, and display processing, diagnosis information (e.g., a subject ID, doctor's observation, etc.), and a diagnosis protocol. Further, according to the need, the storage section 17 is used for storing images that the image database 15 stores. Further, the storage section 17 stores various information for use in the processing performed by the CPU 16.

The interface section 18 is an interface for exchanging various information between the main body 100 and the operation section 19, the position information acquisition section 20, and the network 22. The operation section 19 is provided with an input device such as various switches, a keyboard, a track ball, a mouse, or a touch command screen. The operation section 19 receives various setting requests from an operator and transfers the various setting requests to the main body 100. For example, the operation section 19 receives various operations related to alignment between the ultrasonic image and X-ray CT image.

The monitor 21 displays a GUI (Graphical User Interface) for the operator of the ultrasonic diagnosis apparatus 10 to input various setting requests through the operation section 19 and displays the ultrasonic image and X-ray CT image which are generated in the main body 100 in parallel.

Further, the CPU 16 exchanges three-dimensional image data with the medical image diagnosis apparatus 24 (X-ray CT apparatus 202, MRI apparatus 203, etc.) over the network 22 according to, e.g., DICOM (Digital Imaging and Communications in Medicine) protocol. Note that a configuration may be possible, in which the three-dimensional data obtained by the X-ray CT apparatus and MRI apparatus are stored in a storage medium such as a CD, a DVD, or a USB and then loaded therefrom into the ultrasonic diagnosis apparatus 10.

The position information acquisition section 20 acquires position information indicating a position of the ultrasonic probe 11. For example, as the position information acquisition section 20, a magnetic sensor, an infrared-ray sensor, an optical sensor, or a camera can be used. In the following description, the magnetic sensor is used as the position information acquisition section 20.

The following describes the position information acquisition section 20. In the embodiment, the position information acquisition section 20 is provided in order to align a cross section of the subject's body to be scanned by the ultrasonic probe 11 and a reference image (CT image or MRI image in which a lesion is detected).

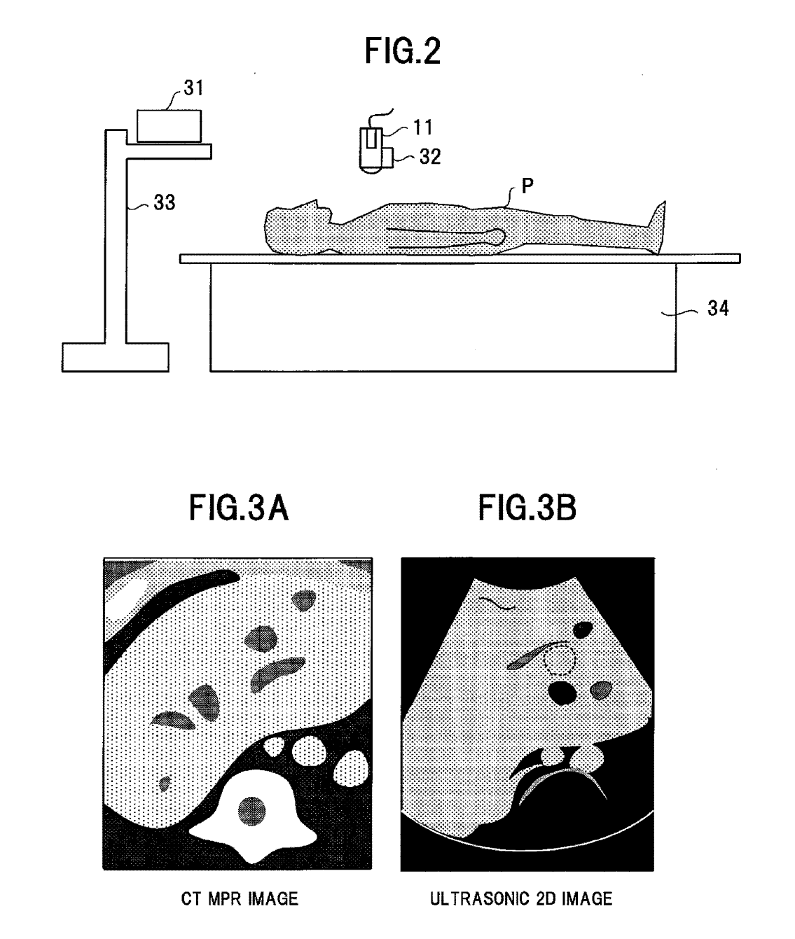

FIG. 2 is an explanatory view schematically illustrating an arrangement of a position sensor of the position information acquisition section 20. That is, a position sensor system of FIG. 2 includes a transmitter 31 and a position sensor (receiver) 32. The transmitter 31 is, e.g., a magnetic transmitter. The transmitter 31 is mounted to a pole 33 set at a fixed position near a bed 34 and transmits a reference signal to form a magnetic field extending outward therearound. Note that the transmitter 31 may be mounted to a leading end of an arm fixed to the ultrasonic diagnosis apparatus main body, or may be mounted to a leading end of an arm of a movable pole stand.

In a three-dimensional magnetic field formed by the transmitter 31, the position sensor 32, which is, e.g., a magnetic sensor, is set within a region where it can receive the magnetism transmitted from the transmitter 31. In the following description, the position sensor 32 is sometimes referred to merely as “sensor 32”.

The sensor 32 is mounted to the ultrasonic probe 11 and receives the reference signal from the transmitter 31 to acquire position information in a three-dimensional space to thereby detect a position and an attitude (inclination) of the ultrasonic probe 11. The position information acquired by the sensor 32 is supplied to the CPU 16 via the interface section 18.

When the subject is scanned by the ultrasonic probe 11, the CPU 16 aligns an arbitrary cross section in the three-dimensional image data generated by the medical image diagnosis apparatus 24 and a cross section to be scanned by the ultrasonic probe 11 to thereby associate the three-dimensional image data with the three-dimensional space.

For example, the CPU 16 calculates, based on a detection result from the sensor 32 mounted to the probe 11, to what position and angle of a subject P an ultrasonic image (two-dimensional image) currently being displayed corresponds. At this time, the transmitter 31 serves as a reference of position/angle information (origin of a coordinate system). Further, the CPU 16 loads volume data of the CT image or MRI image into the ultrasonic diagnosis apparatus 10 to display an MPR image.

The CPU 16 displays the reference image (MPR image) and ultrasonic image on the same screen and performs, for the position alignment, angle alignment that aligns a scanning direction of the ultrasonic probe 11 with a direction corresponding an orientation of the cross section of the reference image and mark alignment that aligns points set on marks observable in both the reference and ultrasonic images with each other. That is, associating the direction and coordinates of the position sensor 32 with coordinates of the volume data allows a two-dimensional image of substantially the same position as the current scanning surface of the ultrasonic probe 11 to be generated from the volume data obtained by another modality, thereby allowing an MPR image of the same cross section as that of the ultrasonic image changing with moving of the ultrasonic probe 11 to be displayed.

With this configuration, afterward, the same cross section as that of the ultrasonic image changing with movement of the ultrasonic probe 11 can be displayed on the MPR image. Thus, a tumor that is difficult to confirm on the ultrasonic image (echo image) can be confirmed on the MPR image. In the following description, the function of thus aligning/combining the ultrasonic image (echo image) and reference image and displaying the aligned/combined image is referred to as “fusion” function.

FIGS. 3A and 3B illustrate a reference and ultrasonic images after alignment, respectively. For example, as the reference image of FIG. 3A, an MPR image (multiple slices image) generated from the volume data collected by an X-ray CT apparatus is used. Alternatively, an image obtained by an MRI apparatus can be used as the reference image.

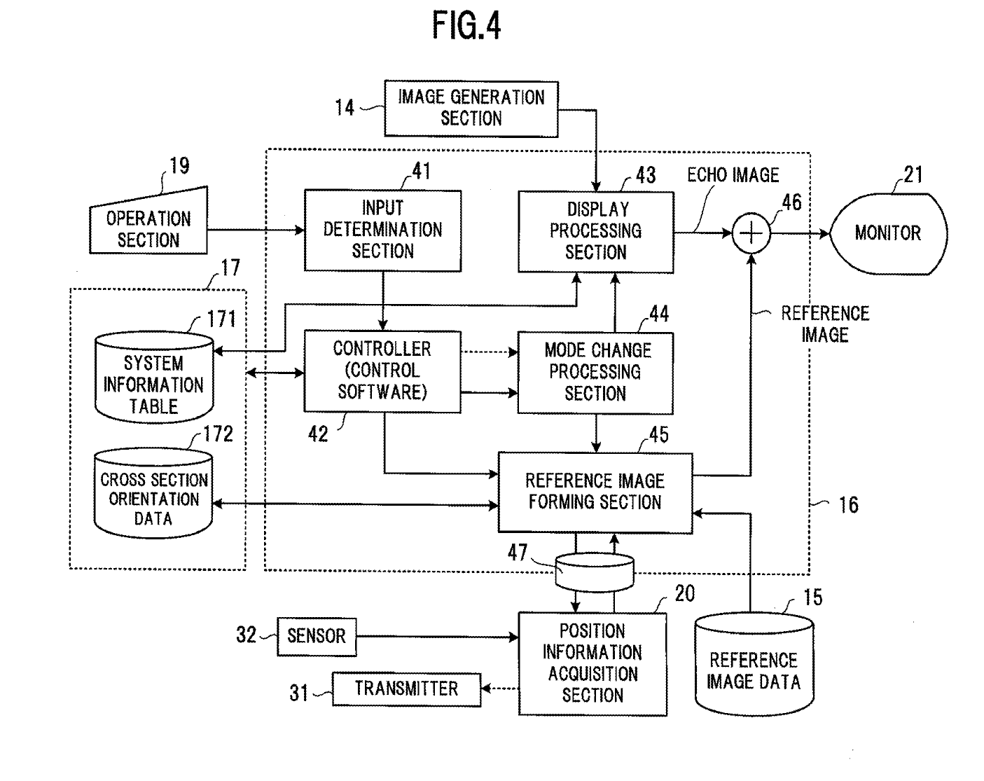

FIG. 4 is a block diagram illustrating a configuration of the CPU 16 which is a characteristic part of the embodiment and components around the CPU 16. As illustrated in FIG. 4, the CPU 16 includes an input determination section 41, a controller 42 including control software, a display processing section 43, a mode change processing section 44, a reference image forming section 45, and a synthesis section 46. The storage section 17 includes a system information table 171 storing therein information related to a type of probes to be selected and information to be used for an examination purpose and a database 172 storing therein cross section orientation data. The image database 15 stores therein three-dimensional images of the CT image or MRI image obtained from the medical image diagnosis apparatus 24.

Information writing and reading in and from the storage section 17 is controlled by the controller 42, and the system information table 171 and the database 172 are connected, respectively, to the display processing section 43 and the reference image forming section 45. The image database 15 is connected to the reference image forming section 45.

The input determination section 41 is connected to the operation section 19. The input determination section 41 determines what kind of input operation has been made on the operation section 19 and supplies determination information to the controller 42. The controller 42 is connected to the mode change processing section 44 and the reference image forming section 45, and the mode change processing section 44 is connected to the display processing section 43 and the reference image forming section 45. The reference image forming section 45 is connected to the position information acquisition section 20 by a cable 47. The reference image formed by the reference image forming section 45 and echo image processed by the display processing section 43 are synthesized in the synthesis section 46, and the synthesized image is output to the monitor 21.

The following describes the fusion function of displaying the ultrasonic image and reference image (e.g., CT image) under control of the CPU 16.

In general, the fusion function is applied in a state where the ultrasonic probe 11 is put on a body surface. The examination purpose of the fusion function is mainly an abdominal area and, more particularly, a liver.

However, when the examination purpose is a prostate gland, two examination methods are available. The first method is a method in which the probe is put on the subject body, like a conventional examination for the abdominal area, and this method is mainly used for observing enlarged prostate. A probe to be used is a convex probe for body surface (e.g., Toshiba PVT-375BT).

The second method is a method in which the probe is inserted from an anus so as to observe the prostate gland through a wall surface of a rectum, and this method is mainly used for observing prostate cancer. Note that the second method may be used for observing the enlarged prostate. A probe to be used is an intracavity convex probe (e.g., Toshiba PVT-781VT).

In a case where the examination purpose is the prostate gland, the MRI image or CT image is often used as the reference image of the fusion function, and “axial” is often used as the orientation of the cross section of the reference image. That is, in the fusion function, the reference image and the echo image need to be aligned with each other in terms of both the angle and position in initial alignment therebetween, and in this case, the “axial” cross section is often used as a reference for user's easy understanding.

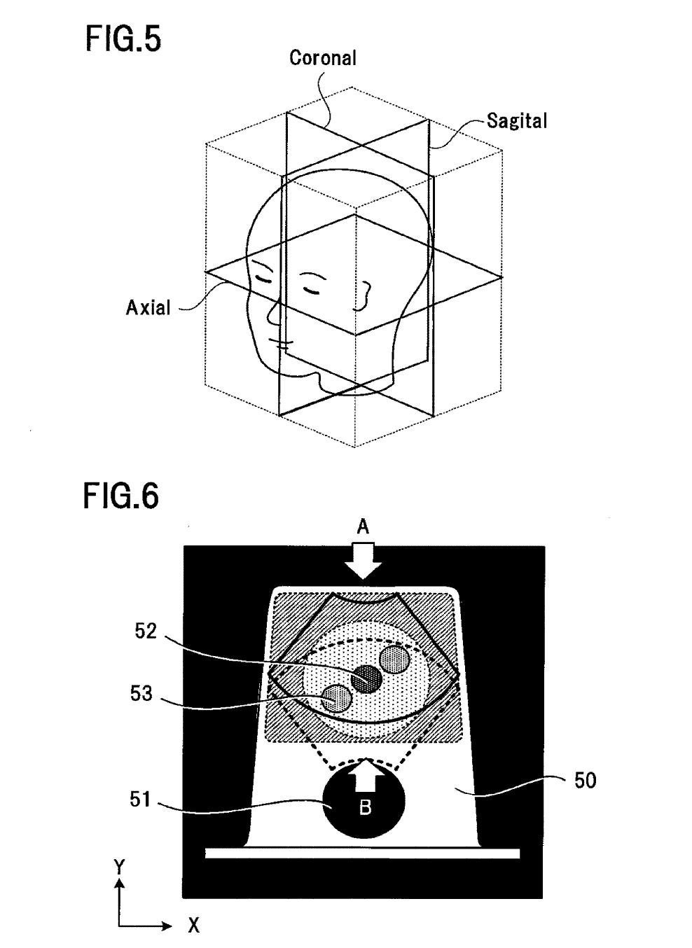

FIG. 5 is an explanatory view schematically illustrating cross section orientations in the CT apparatus or MRI apparatus. As the cross section orientations, there are generally known reference cross section orientations such as a body axis cross section (“axial”) which is a horizontal cross section of the subject, a vertically cut cross section (“sagittal”), and a horizontally cut cross section (“coronal”).

FIG. 6 is an explanatory view illustrating an examination of the prostate gland, in which a phantom is used in place of a subject for descriptive convenience, and the axial cross section of a CT image 50 of the phantom is illustrated. In FIG. 6, a reference numeral 51 denotes a rectum hole, 52 denotes an urethra, and 53 denotes a tumor. As illustrated in FIG. 6, in the examination of the enlarged prostate, the probe is put on a body surface (in an arrow A direction) as denoted by a thick solid line for ultrasonic photographing. On the other hand, in the examination of the prostate cancer, the probe is inserted into the body cavity from the rectum in an arrow B direction as denoted by a thick dashed line for ultrasonic photographing.

In FIG. 6, the orientation of the cross section of the reference image formed by the CT image 51 is “axial”; however, a positional relationship of the objects to be observed in the obtained echo image differs between a case where the ultrasonic probe 11 is put on the body surface and a case where the ultrasonic probe 11 is inserted into the body cavity. Thus, in the case where observation is made from the rectum wall using the probe in the body cavity, the direction of the axial cross section of the reference image and direction of the axial cross section of the echo image may be opposite to each other.

That is, although the alignment with the axial cross section of the reference image is generally performed in the state where the ultrasonic probe 11 is put on the body surface, when the intracavity probe is used to perform observation from the rectum wall, the direction of the axial cross section of the reference image is opposite to the direction of the axial cross section of the thus observed echo image. Thus, in a conventional approach, the reference image is rotated to make the alignment between the reference and echo image.

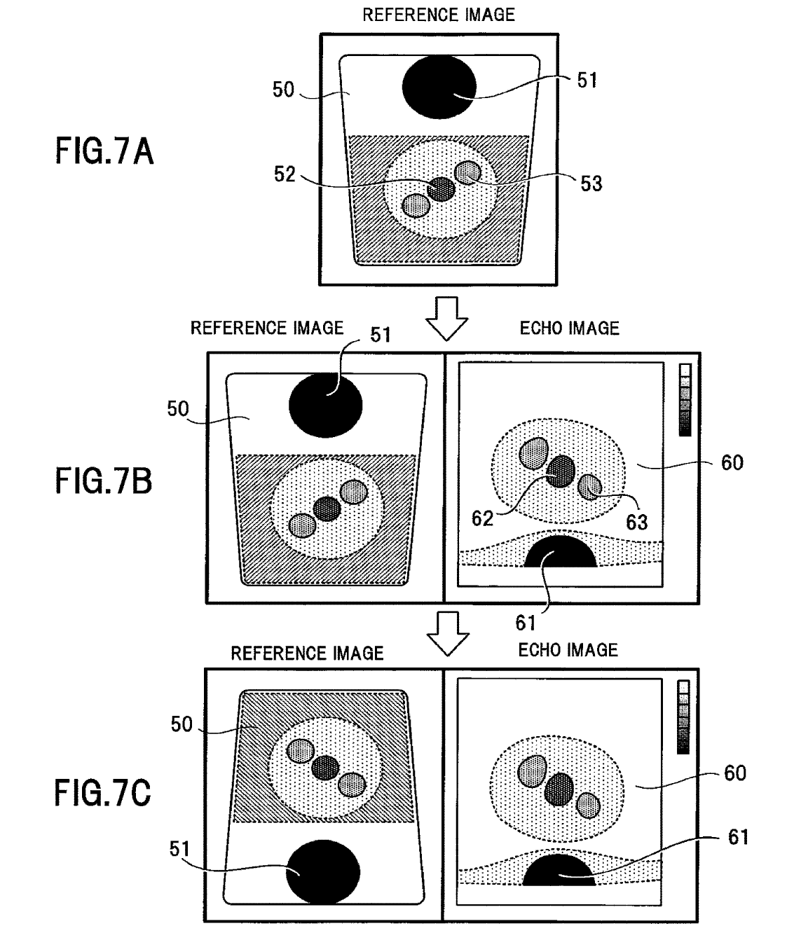

FIGS. 7A to 7C are explanatory views illustrating general rotating processing of the reference image. FIG. 7A illustrates a reference image 50 (CT image) loaded into the image database 15. Upon activation of the fusion function, the reference image 50 and an echo image 60 photographed by an ultrasonic apparatus are displayed in parallel as illustrated in FIG. 7B. In the echo image 60, a reference numeral 61 denotes a rectum hole, 62 denotes an urethra, and 63 denotes a tumor. When the reference image 50 and the echo image 60 are vertically opposite to each other, the reference image 50 is rotated by 180° with respect to an X-axis as illustrated in FIG. 7C.

However, this rotating processing of the image needs to be performed every time the fusion function is used for a new patient, thus taking much time and labor. This imposes a burden on an operator (doctor, laboratory technician, etc.).

Further, when the probe 11 is inserted into the rectum from the anus, an operation direction of the probe is restricted because of a structure of the human body, so that an insertion angle is inclined to some degree with respect to the axial axis (for example, about 30°). Therefore, when the reference image is rotated in the examination of the prostate gland, the reference image is preferably rotated by 150° (=180°−30°).

Thus, in the embodiment, the direction of the cross section of the reference image is initially set according to the examination purpose (prostate gland, heart, internal organs, etc.). Besides, when the reference image needs to be rotated, the rotation angle of the reference image is initially set according to a type (probe for body surface, intracavity convex probe) of the ultrasonic probe 11.

According to the embodiment, by inputting the examination purpose and probe type through the operation section 19 prior to the examination, it is possible to automatically adjust the orientation of the cross section and rotation angle of the reference image according to the initial setting and to display the thus generated reference image together with the echo image.

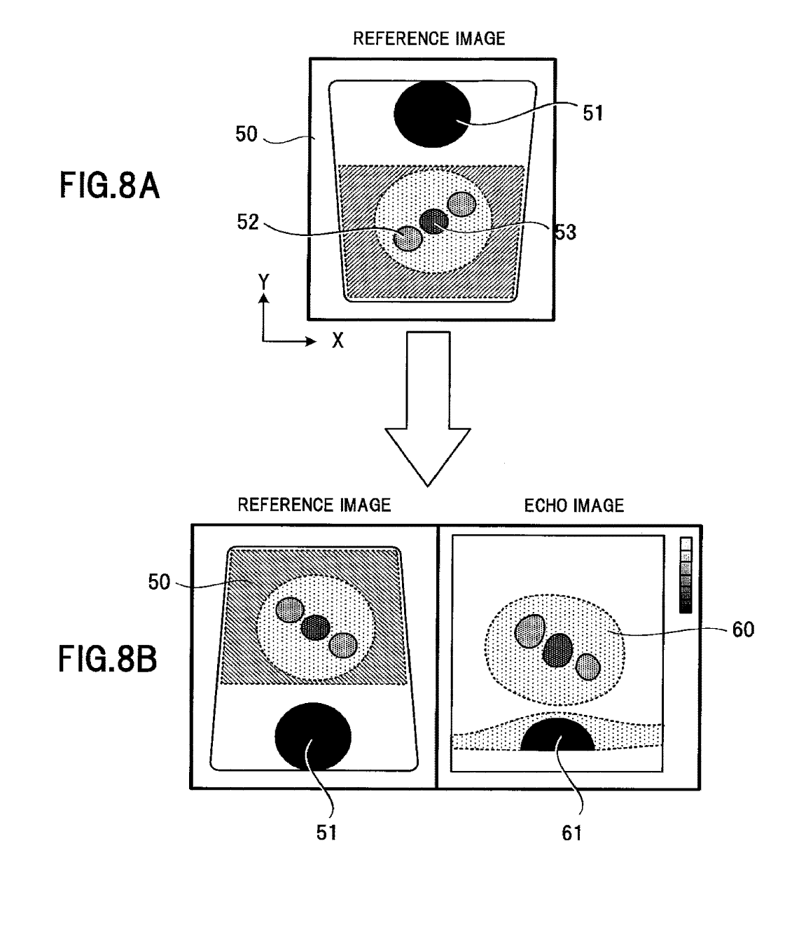

FIGS. 8A and 8B are explanatory views illustrating the rotation processing of the reference image in the embodiment. FIG. 8A illustrates a reference image 50 (CT image) loaded into the image database 15. When the fusion function is activated, the reference image 50 and an echo image 60 photographed by an ultrasonic apparatus are displayed in parallel as illustrated in FIG. 8B. In this state, as the reference image 50, an image of the axial cross section, obtained by rotating the original image by 150° with respect to an X-axis is displayed according to the initial setting. This can eliminate the need for the operator to adjust the reference image many times, thereby reducing time and effort of the operator.

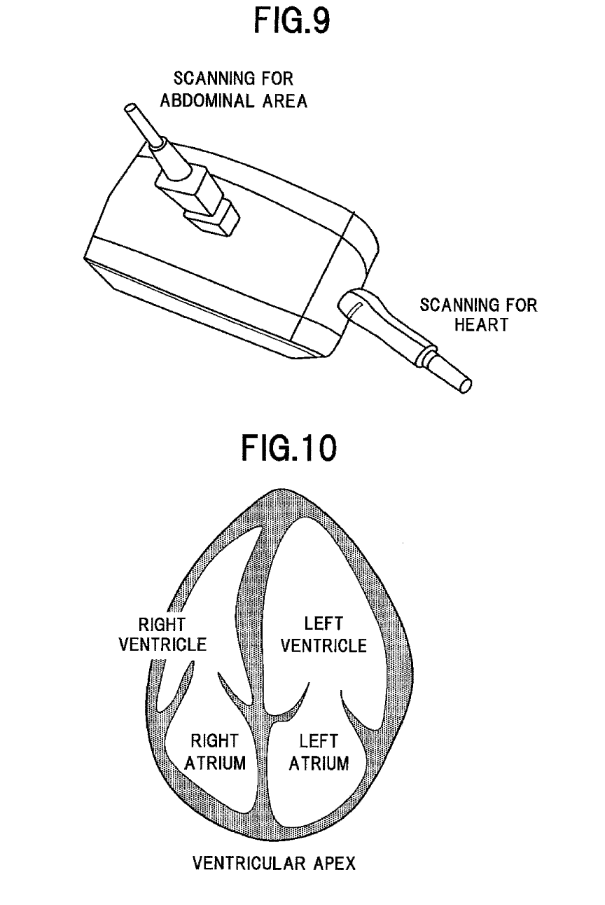

Further, when a plurality of regions are examined with a single probe, it is preferable to change the orientation of the cross section of the reference image according to the examination purpose. For example, as illustrated in FIG. 9, when a sector probe is used, the probe is generally put in a direction corresponding to the axial cross section for scanning of an abdominal area; on the other hand, for scanning of the heart, it is easier to perform examination by setting an apical four-chamber cross section as the reference plane than by setting the axial cross section as the reference plane.

As illustrated in FIG. 10, the apical four-chamber cross section is a cross section suitable for examining presence/absence of abnormality of individual right atrium/right ventricle and left atrium/left ventricle. In this examination, the scanning is performed by the probe such that the four chambers, including a ventricular apex are depicted simultaneously. That is, the orientation of the cross section of the reference image is set so as to correspond to the apical four-chamber cross section, and whereby when the fusion function is activated, a reference image suitable for the examination can be displayed.

In the embodiment, the system information table 171 and the database 172 storing therein the cross section orientation data, which are illustrated in FIG. 4, are added to the configuration of the ultrasonic diagnosis apparatus 10. Further, a function of controlling an initial cross section of the reference image and processing of changing the cross section orientation data of the initial cross section of the reference image according to a button operation of the operator are added to the reference image forming section 45.

The following describes operation of the CPU 16 of FIG. 4. That is, when the operator inputs the examination purpose and type of the ultrasonic probe to be used through the operation section 19, the controller 42 sets the cross section orientation of the reference image according to the input the examination purpose and probe type and stores the cross section orientation data in the database 172. That is, the controller 42 constitutes a cross section orientation setting section.

For example, for a convex probe for body surface, the orientation of the reference image cross section is set to the “axial”, and the rotation angle of the cross section need not be performed (angle after correction=0). For an intracavity convex probe, the orientation of the reference image cross section is set to the “axial”, and a correction angle of 150° in a vertical direction is set for the rotation angle of the cross section. The angle after correction of 150° is an angle obtained by subtracting 30° which is an inclination angle of the probe 11 with respect to the axial plane from the rotation angle of 180°. The rotation in the vertical direction corresponds to a rotation about an X-axis (horizontal axis) in a graphics coordinate system, so that the angle after the correction of 150° is some times referred to as “X-axis rotation amount of 150°”.

In such a state, the operator selects a reference image to be used in the fusion function and then operates the operation section 19 to depress a fusion button so as to start the fusion function. The depression of the button is detected by the input determination section 41. The input determination section 41 checks an operation state of all the buttons provided in the operation section 19 at regular intervals. Thus, the input determination section 41 can determine a state change occurring due to depression of the fusion button and notifies the controller 42 of information indicating that the fusion button has been depressed.

In response to the depression of the fusion button, the controller 42 passes information indicating the probe type and information indicating the examination purpose from the system information table 171 to the mode change processing section 44. The mode change processing section 44 passes, to the reference image forming section 45, information indicating that it is necessary to display the reference image in association with the mode change, layout information of the monitor 21 for displaying the reference image, information related to a display direction of the echo image, and information indicating the probe type and the examination purpose.

The reference image forming section 45 reads a plurality of slice images obtained by, e.g., an MRI apparatus from the image database 15 to thereby construct three-dimensional data. Then, based on the information indicating the probe type and the examination purpose, the reference image forming section 45 acquires, from the database 172, the cross section orientation data according to the used probe. For example, when the probe type is the intracavity convex probe, information of [X-axis rotation amount: 150°] is acquired.

The reference image forming section 45 then acquires, with the body surface as a reference, data from the constructed three-dimensional data of the MRI image, sequentially from a position rotated by 150° about the X-axis from a center of the data, thereby constructing a two-dimensional image. The reading start position of the data is a contact position between the probe and subject and, as the reading position advances in a Y-axis direction, images gradually separated from the contact position are sequentially formed to thereby acquire two-dimensional image data.

Further, as illustrated in the right part of FIG. 8B, in a case of the echo image in which the contact position between the probe and subject is located at a lower portion on the monitor, that is, when a vertically inverted image is displayed, the reference image forming section 45 processes the two-dimensional reference image so as to make the direction of the reference image coincide with that of the vertically inverted echo image and outputs the thus processed reference image to the synthesis section 46.

The synthesis section 46 synthesizes the echo image processed by the display processing section 43 and reference image formed by the reference image forming section 45 and outputs the synthesized image to the monitor 21. As illustrated in FIG. 8B, the processed echo image and reference image are displayed in parallel on the monitor 21.

When the operator changes the inclination of the reference image, information indicating the inclination change is transmitted from the operation section 19 to the controller 42 through the input determination section 41. Then, the controller 42 transmits information related to a rotation axis and rotation amount to the reference image forming section 45. Then, based on the information related to a rotation axis and rotation amount, the reference image forming section 45 constructs the two-dimensional reference image from the three-dimensional data and outputs the constructed reference image.

Further, when the operator depresses a storage button in the operation section for the purpose of storing the changed display direction, information indicating that the storage button has been depressed is transmitted to the reference image forming section 45 through the input determination section 41 and controller 42. Then, the reference image forming section 45 updates and stores, in the database 172, the cross section orientation data corresponding to the information related to the selected probe type. Note that when the examination purpose is the heart, the reference image is displayed such that the orientation of the cross section of the reference image corresponds to the apical four-chamber cross section.

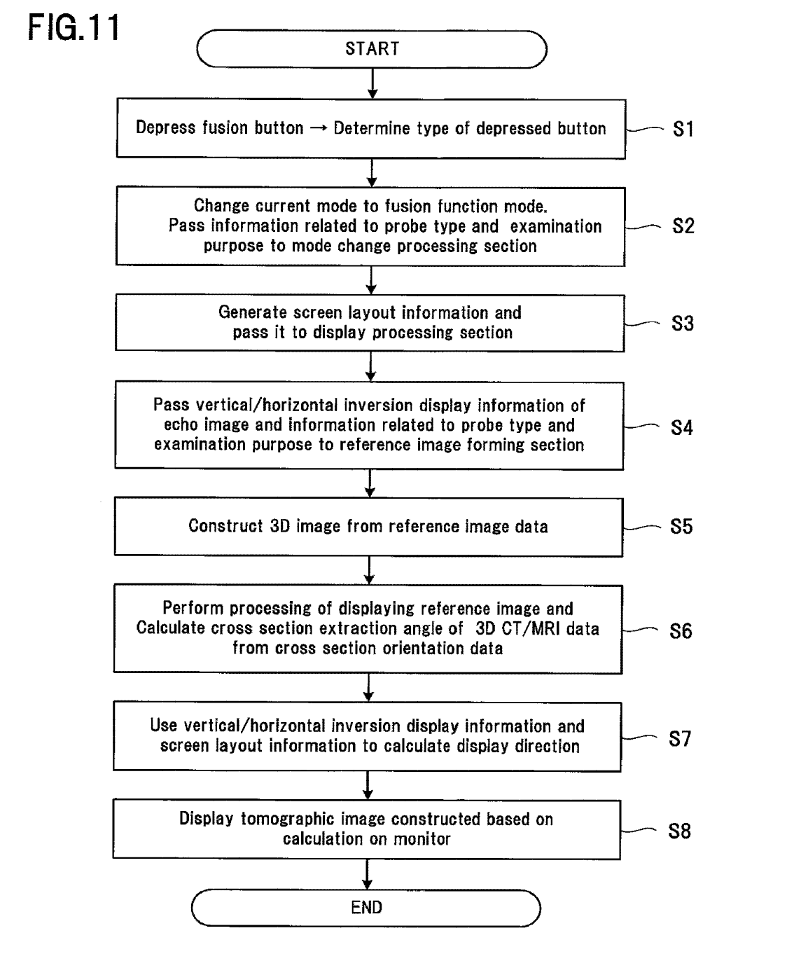

FIG. 11 is a flowchart explaining the operation of the CPU 16 of FIG. 4. It is assumed that the operator selects the reference image to be used in the fusion function in a start step of FIG. 11. In step S1, the operator operates the operation section 19 to depress the fusion button. Then, the input determination section 41 determines a type of the depressed button and provides corresponding information to the controller 42.

In step S2, the controller 42 instructs, based on the information from the input determination section 41, the mode change processing section 44 to change a current mode to the fusion function mode. Further, the controller 42 passes, to the mode change processing section 44, the information related to the examination purpose and selected probe type stored in the system information table 171.

In the next step S3, the mode change processing section 44 generates screen layout information associated with the mode change and passes the generated information to the display processing section 43. In step S4, the mode change processing section 44 passes vertical/horizontal inversion display information of the echo image and information related to the probe type and the examination purpose to the reference image forming section 45.

In step S5, the reference image forming section 45 constructs a three-dimensional image based on the reference image data read from the image database 15. Further, in step S6, the reference image forming section 45 performs processing of displaying the reference image and calculates, based on the information related to the probe type and the examination purpose, a cross section extraction angle of the three-dimensional CT/MRI image data from the cross section orientation data read from the database 172. Further, in step S7, the reference image forming section 45 uses the vertical/horizontal inversion display information and screen layout information to calculate the display direction of the image.

Then, in step S8, the reference image forming section 45 forms the tomographic image constructed based on the calculation performed in steps S6 and S7 as the reference image, outputs the reference image to the synthesis section 46, displays the reference image on the monitor 21, and ends this routine.

As described above, in the embodiment, the cross section orientation of the reference image is set according to the examination purpose for the subject and type of the ultrasonic probe to be used, so that it is possible to set the cross section of the reference image in a desired direction before alignment with the echo image. Thus, the operator can display an ultrasonic image and its corresponding reference image simply by depressing the fusion button. That is, the operation procedure can be simplified.

Further, even in a case where the operation direction of the probe is restricted (for example, when the ultrasonic probe to be used is an intracavity probe), it is possible to rotate the reference image to a desired angle, thereby displaying an image suitable for examination.

While certain embodiments have been described, these embodiments have been presented by way of example only, and are not intended to limit the scope of the invention. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. An ultrasonic diagnosis apparatus comprising:
an ultrasonic image generation section that generates an ultrasonic image based on a reception signal from an ultrasonic probe that transmits an ultrasonic wave to a subject and receives the ultrasonic wave from the subject;
a position information acquisition section that includes a position sensor mounted to the ultrasonic probe and acquires position information on a three-dimensional space of the ultrasonic probe;
an image acquisition section that obtains image data and acquires a reference image corresponding to the ultrasonic image;
a reference image forming section that identifies a to-be-displayed cross section orientation of acquired the reference image according to at least one of information related to an examination purpose for the subject and information related to a type of the ultrasonic probe, and forms a reference image which a cross section orientation is identified; and
a display section that displays a formed reference image by the reference image forming section and ultrasonic image formed by the ultrasonic image generation section.

2. The apparatus of claim 1, wherein
the reference image forming section includes a storage section that stores data of the identified cross section orientation.

3. The apparatus of claim 2, wherein
the reference image forming section updates the cross section orientation data stored in the storage section, when an orientation of a cross section of the formed reference image is changed by an operator.

4. The apparatus of claim 1, wherein
the reference image forming section performs alignment between the ultrasonic image and the image data based on the position information and generates, from the image data, a two-dimensional reference image that corresponds to a scanning position of the ultrasonic probe and that is automatically adjusted in terms of the cross section orientation based on the cross section orientation data.

5. The apparatus of claim 1, wherein
the reference image forming section identifies, as the to-be-displayed cross section orientation of the formed reference image, any one of “axial”, “sagittal”, and “coronal”.

6. The apparatus of claim 4, wherein
the reference image forming section further sets a rotation angle of the two-dimensional reference image according to the information of the examination region and information of the probe type and adjusts the rotation angle of the two-dimensional reference image based on the set rotation angle.

7. The apparatus of claim 1, wherein
the reference image forming section identifies the to-be-displayed cross section orientation of the formed reference image according to an ultrasonic probe for body surface and an intracavity ultrasonic probe.

8. The apparatus of claim 1, wherein
the reference image forming section identifies the to-be-displayed cross section orientation of the formed reference image according to the examination region, when different regions are examined with a single probe.

9. The apparatus of claim 1, wherein
the reference image forming section makes the cross section orientation of the formed reference image coincide with an apical four-chamber cross section, when the examination region using the ultrasonic probe is a heart.

10. The apparatus of claim 1, wherein
the image acquisition section acquires, as the image data, three-dimensional image data photographed by a medical image diagnosis apparatus other than the ultrasonic diagnosis apparatus.
